(19) United States
(12) Patent Application Publication
(10) Pub. No.: US 2010/0168445 A1
(43) Pub. Date: Jul. 1, 2010

(54) PROCESS FOR PREPARING EPSILON-CAPROLACTONE

(75) Inventors: Rolf Pinkos, Bad Duerkheim (DE); Gerd Tebben, Mannheim (DE); Christophe Bauduin, Mannheim (DE); Daniel Breuninger, Bobenheim-Roxheim (DE); Maria Guixa Guardia, Mannheim (DE); Tilman Sirch, Schifferstadt (DE); Thomas Krug, Worms (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, LUDWIGSHAFEN (DE)

(21) Appl. No.: 12/602,837

(22) PCT Filed: Jun. 6, 2008
(86) PCT No.: PCT/EP08/57114

§ 371 Date: Dec. 3, 2009

(30) Foreign Application Priority Data
Jun. 14, 2007 (EP) 07110270.1

(51) Int. Cl. C07D 313/04

(52) U.S. Cl. 549/266

(57) ABSTRACT

The present invention provides a process for preparing ε-caprolactone in a purity above 99%, in which 6-hydroxycaproic ester comprising from 0.5 to 40% by weight of adipic diester is cyclized in the gas phase at from 150 to 450° C. in the presence of oxidic catalysts and ε-caprolactone is obtained from the cyclization product by distillation.

The invention relates to a preparation of ε-caprolactone in a purity above 99%, which comprises cyclizing 6-hydroxycaproic ester comprising from 0.5 to 40% by weight of adipic diester in the gas phase at from 150 to 450° C. in the presence of oxidic catalysts and obtaining ε-caprolactone from the cyclization product by distillation.

ε-caprolactone and the polycaprolactones prepared therefrom by polyaddition serve to prepare polyurethanes.

The aqueous solutions of carboxylic acids which are formed as by-products in the oxidation of cyclohexane to cyclohexanol and cyclohexanone (cf. Ullmann's Encyclopedia of Industrial Chemistry, 5th ed., 1987, vol. A8, p. 49), referred to hereinafter as dicarboxylic acid solution (DCS), comprise (calculated in anhydrous form in % by weight) generally between 10 and 40% adipic acid, between 10 and 40% 6-hydroxycaproic acid, between 1 and 10% glutaric acid, between 1 and 10% 5-hydroxyvaleric acid, between 1 and 5% 1,2-cyclohexanediols, between 1 and 5% 1,4-cyclohexanediols, between 2 and 10% formic acid, and a multitude of further mono- and dicarboxylic acids, esters, oxo and oxa compounds whose individual contents generally do not exceed 5%. Examples include acetic acid, propionic acid, butyric acid, valeric acid, caproic acid, oxalic acid, malonic acid, succinic acid, 4-hydroxybutyric acid and ε-butyrolactone.

The preparation of caprolactone from DCS has also already been described, for example, in DE 1 618 143. In this preparation, dewatered DCS is reacted thermally with phosphoric acid, and a mixture of dicarboxylic acids, caprolactone and a multitude of other components is fractionated. The bottoms are obtained partly in solid and sparingly soluble form. However, the caprolactone, even after further distillative workup, has only a 98% purity.

Also described in DE 38 23 213 is the conversion of 6-hydroxycaproic ester in the gas phase in the presence of oxidic catalysts and of an inert carrier gas to caprolactone.

Moreover, WO 97/31 883 describes a process for preparing 1,6-hexanediol and ε-caprolactone from a carboxylic acid mixture which comprises adipic acid, 6-hydroxycaproic acid and small amounts of 1,4-cyclohexanediols and is obtained as a by-product of the oxidation of cyclohexane to cyclohexanone/cyclohexanol with oxygen or oxygen-comprising gases and by water extraction of the reaction mixture, which is esterified with a low molecular weight alcohol to the corresponding carboxylic esters, the resulting esterification mixture is freed of excess alcohol and low boilers with a first distillation stage, the bottom product is separated in a second distillation stage into an ester fraction essentially free of 1,4-cyclohexanediols and a fraction comprising at least the majority of the cyclohexanediols, and a fraction comprising essentially 6-hydroxycaproic acid (stage 12) is obtained by a third distillation stage and is cyclized to ε-caprolactone in the gas or liquid phase.

Since the boiling ranges of adipic esters and 6-hydroxycaproic esters barely differ, the two substances can generally be obtained without the other in each case only with extremely high distillation complexity, for example by using columns with very high numbers of separating stages and a correspondingly high energy demand, or by adding an extraneous substance which has a boiling point between the two esters.

In order to reduce the separation complexity and in order to obtain pure 6-hydroxycaproic ester, the distillative separation of the two C₆-esters in the third distillation stage according to WO 97/31 883 has to date been performed such that the adipic diester to be hydrogenated to 1,6-hexanediol still comprised from 0.2 to 7% by weight of 6-hydroxycaproic ester. In the case of a high demand for 1,6-hexanediol, it is also possible to remove even more 6-hydroxycaproic ester together with adipic diester and to hydrogenate it to 1,6-hexanediol with further reduction in the separation complexity. The 6-hydroxycaproic ester content of the dicarboxylic acid solution has therefore to date never been utilized completely for caprolactone preparation.

When the utilization of the majority or of the entirety of the 6-hydroxycaproic ester for caprolactone preparation is desired without an extremely high level of distillation complexity or the addition of an extraneous substance, the cyclization of the 6-hydroxycaproic ester stream has to be possible in the presence of relatively large amounts of adipic ester without disadvantages.

WO 97/31 883 recommends the preparation of caprolactone in the liquid phase. According to comparative example 1 present in this application, however, a significant decline in the yield of caprolactone is observed for the cyclization in the liquid phase in the presence of 5% by weight of adipic ester based on the 6-hydroxycaproic ester.

This decline in yield is attributable to polymerization side reactions in the ε-caprolactone cyclization. In the presence of catalysts, dimers, oligomers and polymers can form from adipic diesters and 6-hydroxycaproic esters. Dimethyl adipate and methyl 6-hydroxycaproate can form, for example, the dimeric ester CH₃OOC—(CH₂)₄—COO—(CH₂)₅—COOCH₃which can form oligomers or polymers with incorporation of further 6-hydroxycaproic esters. Although these dimers, oligomers or polymers are compounds still utilizable by hydrogenation for 1,6-hexanediol, the risk of deposits of these high-boiling components on the cyclization catalyst is great in the case of reactions in the gas phase, such that a very shortened catalyst lifetime would have to be expected.

Moreover, it was known from EP-A 251 111 that adipic diesters can be converted to cyclopentanones in the presence of catalysts and are thus no longer available for other applications, for example the conversion of 1,6-hexanediol.

It was therefore an object of the invention to provide a process for preparing caprolactone in a purity of more than 99% proceeding from dicarboxylic esters or mixtures thereof, which is accompanied by a reduction in the separation complexity and the utilization of the majority or of the entirety of the 6-hydroxycaproic ester for caprolactone preparation, and in which good catalyst lifetimes are achieved through avoidance of polymerization side reactions in the ε-caprolactone cyclization. In addition, a minimum amount of adipic ester should be converted, since it should, after removal of caprolactone, as far as possible be available to other applications.

This object is achieved by a process for preparing ε-caprolactone in a purity above 99%, which comprises cyclizing 6-hydroxycaproic ester comprising from 0.5 to 40% by weight, preferably from 0.6 to 25% by weight, more preferably from 0.7 to 15% by weight, of adipic diester in the gas phase at from 150 to 450° C. in the presence of oxidic catalysts and obtaining ε-caprolactone from the cyclization product by distillation.

Useful esterifying alcohols of the 6-hydroxycaproic ester and of the adipic ester generally include alkanols having from 1 to 12 carbon atoms, cycloalkanols having from 5 to 7 carbon atoms, aralkanols having from 7 to 8 carbon atoms or phenols having from 6 to 6 carbon atoms. It is possible to use methanol, ethanol, propanol, isopropanol, n- or i-butanol or else n-pentanol or i-pentanol or mixtures of the alcohols, but preferably alcohols having from 1 to 4 carbon atoms, more preferably methanol. Diols such as butanediol or pentanediol are also useful in principle. The ester groups in the 6-hydroxycaproic esters and the adipic esters may be the same or different, but are preferably the same. The particularly preferred reactant is methyl 6-hydroxycaproate comprising from 0.5 to 40% by weight of dimethyl adipate.

The reactant of the process according to the invention, the 6-hydroxycaproic ester comprising from 0.5 to 40% by weight of adipic ester, can also be prepared according to DE-A 197 50 532, which is hereby explicitly incorporated by reference.

According to DE-A 197 50 532, 6-hydroxycaproic ester comprising from 0.5 to 40% by weight of adipic diester is obtained by catalytic hydrogenation of adipic diesters or reactant streams which comprise these esters as essential constituents, distillation of the hydrogenation effluent and removal of the hexanediol.

The hydrogenation is preferably performed in the liquid phase. The hydrogenation catalysts used in this process are generally heterogeneous, but also homogeneous catalysts suitable for hydrogenating carbonyl groups. They may be used either in fixed-bed or mobile form, for example in a fluidized bed reactor. Examples thereof are described, for example, in Houben-Weyl, Methoden der Organischen Chemie [Methods of Organic Chemistry], volume IV/1c, p. 16 to 26.

Among the hydrogenation catalysts to be used, preference is given to those which comprise one or more elements of group Ib, VIb, VIIb and VIIIb, and also IIIa, IVa and Va of the Periodic Table of the Elements, especially copper, chromium, rhenium, cobalt, rhodium, nickel, palladium, iron, platinum, indium, tin and/or antimony. Particular preference is given to catalysts which comprise copper, cobalt and/or rhenium.

In addition, the 6-hydroxycaproic ester comprising from 0.5 to 40% by weight of adipic diester can be prepared according to WO 97/31 883, which is hereby incorporated explicitly by reference.

The 6-hydroxycaproic ester comprising from 0.5 to 40% by weight of adipic diester is prepared according to WO 97/31 883 by esterifying a carboxylic acid mixture which comprises adipic acid, 6-hydroxycaproic acid and small amounts of 1,4-cyclohexanediols and is obtainable as a by-product of the oxidation of cyclohexane to cyclohexanone/cyclohexanol with oxygen or oxygen-comprising gases by water extraction of the reaction mixture with a low molecular weight alcohol to give the corresponding carboxylic esters, and separating the esterification mixture thus obtained in at least one distillation stage.

In a preferred embodiment, methyl 6-hydroxycaproate comprising from 0.5 to 40% by weight of dimethyl adipate is obtained by
freeing the esterification mixture obtained of excess methanol and low boilers in a first distillation stage,
from the bottom product, in a second distillation stage, performing a separation into ester fraction essentially free of 1,4-cyclohexanediols and a fraction comprising at least the majority of the 1,4-cyclohexanediols,
removing the methyl 6-hydroxycaproate stream comprising from 0.5 to 40% by weight of dimethyl adipate from the ester fraction in a third distillation stage.

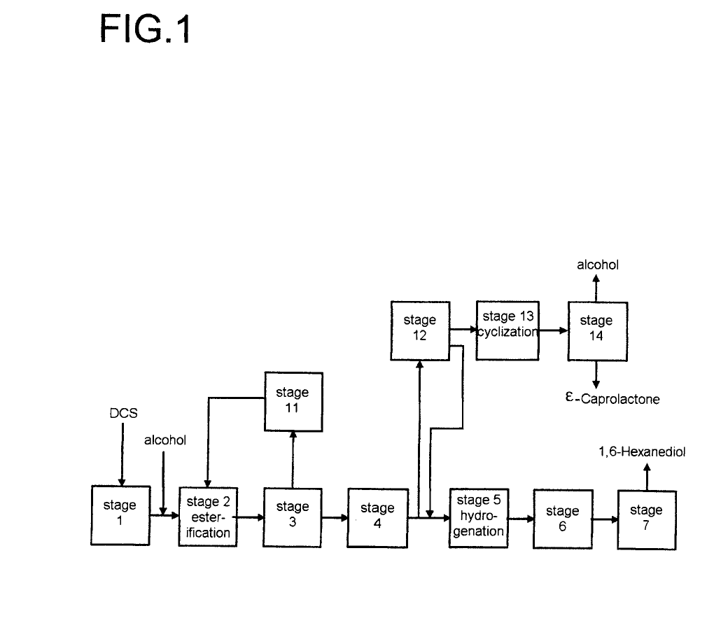

For better understanding, the process for preparing ε-caprolactone is explained according to WO 97/31 883 in FIG. 1, in which the individual process steps are broken down into further stages, of which stages 2, 3, 4 and 12, 13 and 14 are essential for the process for preparing ε-caprolactone, and stages 3 and 4 may also be combined.

The dicarboxylic acid solution (DCS) is generally an aqueous solution having a water content of from 20 to 80%. Since an esterification reaction is an equilibrium reaction in which water forms, it is advisable, especially in the case of esterification with methanol, for example, to remove water from the reaction, in particular when water cannot be removed during the esterification reaction, for example by azeotropic means. The dewatering in stage 1 can be effected, for example, with a membrane system, or preferably by means of a distillation apparatus in which water is removed via the top and higher monocarboxylic acids, dicarboxylic acids and 1,4-cyclohexanediols via the bottom at from 10 to 250° C., preferably from 20 to 200° C., particularly from 30 to 200° C., and a pressure of from 1 to 1500 mbar, preferably from 5 to 1100 mbar, more preferably from 20 to 1000 mbar. The bottom temperature is preferably selected such that the bottom product can be drawn off in liquid form. The water content in the bottom of the column may be from 0.01 to 10% by weight, preferably from 0.01 to 5% by weight, more preferably from 0.01 to 1% by weight.

The water can be removed in such a way that the water is obtained in predominantly acid-free form, or the lower monocarboxylic acids present in the DCS—essentially formic acid—can be distilled off for the most part with the water in order that they do not bind any esterification alcohol in the esterification.

Alcohol ROH having from 1 to 10 carbon atoms can also be added to the carboxylic acid stream from stage 1. It is possible to use methanol, ethanol, propanol or isopropanol, or mixtures of the alcohols, but preferably methanol, on the one hand, or C₄ and higher alcohols, especially having from 4 to 8 carbon atoms and preferably n- or i-butanol or else n-pentanol or i-pentanol on the other hand. The mixing ratio of alcohol to carboxylic acid stream (mass ratio) may be from 0.1 to 30, preferably from 0.2 to 20, more preferably from 0.5 to 10.

This mixture passes as a melt or solution into the reactor of stage 2, in which the carboxylic acids are esterified with the alcohol. The esterification reaction can be performed at from 50 to 400° C., preferably from 70 to 300° C., more preferably from 90 to 200° C. It is possible to apply an external pressure, but preference is given to performing the esterification reaction under the autogenous pressure of the reaction system. The esterification apparatus used may be one stirred tank or flow tube, or it is possible in each case to use a plurality. The residence time needed for the esterification is between 0.3 and 10 hours, preferably from 0.5 to 5 hours. The esterification reaction can proceed without addition of a catalyst, but preference is given to increasing the reaction rate by adding a catalyst. The catalyst may be a homogeneously dissolved catalyst or a solid catalyst. Examples of homogeneous catalysts include sulfuric acid, phosphoric acid, hydrochloric acid, sulfonic acids such as p-toluenesulfonic acid, heteropolyacids such as tungstophosphoric acid, or Lewis acids, for example aluminum, vanadium, titanium, boron compounds. Preference is given to mineral acids, especially sulfuric acid. The weight ratio of homogeneous catalyst to carboxylic acid melt is generally from 0.0001 to 0.5, preferably from 0.001 to 0.3.

Suitable solid catalysts are acidic or superacidic materials, for example acidic and superacidic metal oxides such as SiO₂, Al₂O₃, SnO₂, ZrO₂, sheet silicates or zeolites, all of which may be doped with mineral acid residues such as sulfate or phosphate for acid strengthening, or organic ion exchangers with sulfonic acid or carboxylic acid groups. The solid catalysts may be arranged as a fixed bed or be used as a suspension.

The water formed in the reaction is appropriately removed continuously, for example by means of a membrane or by distillation.

The completeness of the conversion of the free carboxyl groups present in the carboxylic acid melt is determined with the acid number measured after the reaction (mg KOH/g). Minus any acid added as a catalyst, it is from 0.01 to 50, preferably from 0.1 to 10. Not all carboxyl groups present in the system need be present as esters of the alcohol used, but rather a portion may be present in the form of dimeric or oligomeric esters with the OH end of the hydroxycaproic acid.

The esterification mixture is fed into stage 3, a membrane system or preferably a distillation column. When a dissolved acid has been used as a catalyst for the esterification reaction, the esterification mixture is appropriately neutralized with a base, in which case from 1 to 1.5 base equivalents are added per acid equivalent of the catalyst. The bases used are generally alkali metal or alkaline earth metal oxides, alkali metal or alkaline earth metal carbonates, alkali metal or alkaline earth metal hydroxides, or alkali metal or alkaline earth metal alkoxides, or amines in substance or dissolved in the esterification alcohol. However, it is also possible to neutralize with basic ion exchangers.

When a column is used in stage 3, the feed to the column is preferably between the top stream and the bottom stream. The excess esterification alcohols ROH, water and corresponding esters of formic acid, acetic acid and propionic acid are drawn off via the top at pressures of from 1 to 1500 mbar, preferably from 20 to 1000 mbar, more preferably from 40 to 800 mbar, and temperatures between 0 and 150° C., preferably 15 and 90° C. and especially 25 and 75° C. This stream can either be incinerated or preferably worked up further in stage 11.

The bottoms obtained are an ester mixture which consists predominantly of the esters of the alcohol ROH used with dicarboxylic acids such as adipic acid and glutaric acid, hydroxycarboxylic acids such as 6-hydroxycaproic acid and 5-hydroxyvaleric acid, and oligomers and free and esterified 1,4-cyclohexanediols. It may be advisable to permit a residual content of water and/or alcohol ROH up to 4% by weight in each case in the ester mixture. The bottom temperatures are preferably from 70 to 250° C., preferably from 80 to 220° C., more preferably from 100 to 190° C.

The stream from stage 3 which has been substantially freed of water and esterification alcohol ROH is fed into stage 4. This is a distillation column in which the feed is between the low-boiling components and the high-boiling components. The column is operated at temperatures of from 10 to 300° C., preferably from 20 to 270° C., more preferably from 30 to 250° C., and pressures of from 1 to 1000 mbar, preferably from 5 to 500 mbar, more preferably from 10 to 200 mbar.

The top fraction consists predominantly of residual water and residual alcohol ROH, esters of the alcohol ROH with monocarboxylic acids, preferably C₃- to C₆-mono-carboxylic esters with hydroxycarboxylic acids such as 6-hydroxycaproic acid, 5-hydroxyvaleric acid and in particular the diesters with dicarboxylic acids such as adipic acid, glutaric acid and succinic acid, cyclohexanediols, caprolactone and valerolacetone.

The components mentioned may be removed together via the top or, in a further preferred embodiment, in the column of stage 4 in a top stream which comprises predominantly residual water and residual alcohol and the abovementioned constituents having from 3 to 5 carbon atoms, and the sidestream which comprises predominantly the abovementioned constituents of the C₆ esters. The stream comprising the esters of C₆ acids, either as an overall top stream or as a sidestream, can then, according to how much caprolactone is to be prepared, be fed only partly or as the entire stream into stage 12 in the process preferred according to WO 97/31 883.

The high-boiling components of the stream from stage 4, predominantly consisting of dimeric or oligomeric esters, cyclohexanediols and undefined constituents of the DCLS, some of which are polymeric, are removed via the stripping section of the column of stage 4. may either be incinerated or, in a preferred embodiment for so-called transesterification, pass into the stage 8 described in WO 97/31 883.

Stages 3 and 4 may be combined, especially when only relatively small amounts are processed. To this end, for example, the C₆ ester stream can be obtained in a fractional distillation performed batchwise.

For the caprolactone preparation, the stream from stage 4 comprising predominantly esters of the C₆ acids is used. To this end, this stream is separated in stage 12, a distillation column, into a stream comprising predominantly adipic diester via the top and a stream comprising predominantly 6-hydroxycaproic ester via the bottom. The column is operated at pressures of from 1 to 500 mbar, preferably from 5 to 350 mbar, more preferably from 10 to 200 mbar, and bottom temperatures of from 80 to 250° C., preferably from 100 to 200° C., more preferably from 110 to 180° C. The top temperatures are established correspondingly.

What is important for a high purity and high yield of caprolactone is the removal of the 1,2-cyclohexanediols from the hydroxycaproic ester, since these components form azetropes with one another. It was not foreseeable in this stage 12 that the separation of the 1,2-cyclohexanediols and of the hydroxycaproic ester succeeds completely, in particular when the ester used is the preferred methyl ester.

It may be advantageous also to remove some hydroxycaproic ester in stage 12 together with the adipic diester. The contents in the adipic ester of hydroxycaproic ester are, when the adipic diester is to be hydrogenated to 1,6-hexanediol, advantageously between 0.2 and 7% by weight. According to the alcohol component of the esters, this proportion of hydroxycaproic ester is removed together with the adipic diester via the top (e.g. methyl ester) or via the bottom (e.g. butyl ester).

The stream comprising 6-hydroxycaproic ester having from 0.5 to 40% by weight of adipic diester is converted in the gas phase to alcohol and caprolactone. These mixtures of 6-hydroxycaproic esters and adipic diesters may also comprise further components which may make up a proportion by weight of up to 20%, but preferably a proportion below 10%, more preferably below 5%. These components consist, for example, of 1,5-pentanediol, cyclohexanediols, unsaturated adipic diesters, pimelic diesters, caprolactone, 5-hydroxycaproic ester and diesters based in particular on 6-hydroxycaproic esters.

To this end, the mixture of 6-hydroxycaproic ester and from 0.5 to 40% by weight of adipic diester is passed in vaporous form together with a carrier gas over fixed bed oxidic catalysts or oxidic catalysts present in upward and downward swirling motion.

The evaporation is effected at from 180 to 300° C. It may be advantageous additionally to evaporate a solvent inert under the reaction conditions. Useful such solvents include, for example, ethers such as tetrahydrofuran or dioxane, but also alcohols. Advantageously, from 10 to 95% by weight solutions of 6-hydroxycaproic esters and adipic diesters in such solvents are used as the reactant for the process according to the invention.

Inert carrier gases are, for example, nitrogen, carbon dioxide, hydrogen or noble gases, for example argon. Preference is given to using nitrogen or hydrogen as the carrier gas. In general, from 5 to 100 mol of carrier gas, preferably from 8 to 50 mol, more preferably from 10 to 30 mol, are used per mole of vaporous 6-hydroxycaproic ester. The carrier gas is preferably circulated by means of a blower or a compressor, in which case a substream can be discharged and replaced correspondingly by fresh gas.

The reaction is performed in the presence of a catalyst. Suitable catalysts are acidic or basic catalysts which may be present in homogeneously dissolved or heterogeneous form. Examples are alkali metal and alkaline earth metal hydroxides, alkali metal and alkaline earth metal oxides, alkali metal and alkaline earth metal carbonates, alkali metal and alkaline earth metal alkoxylates, or alkali metal and alkaline earth metal carboxylates, acids such as sulfuric acid or phosphoric acid, organic acids such as sulfonic acids or mono- or dicarboxylic acids, or salts of the aforementioned acids, Lewis acids, preferably from main groups III and IV or of transition groups I to VIII of the Periodic Table of the Elements, or oxides of rare earth metals or mixtures thereof. Examples include magnesium oxide, zinc oxide, boron trioxide, titanium dioxide, silicon dioxide, tin dioxide, bismuth oxide, copper oxide, lanthanum oxide, zirconium dioxide, vanadium oxides, chromium oxides, tungsten oxides, iron oxides, cerium oxide, aluminum oxide, hafnium oxide, lead oxide, antimony oxide, barium oxide, calcium oxide, sodium hydroxide, potassium hydroxide, neodymium oxide. It is also possible to use mixtures of oxides, which may be mixtures of the individual components or else mixed oxides as occur, for example, in zeolites, aluminas or heteropolyacids. To increase the acid strength, the catalysts may have been pretreated, for example with mineral acids, for example with sulfuric acid, phosphoric acid or hydrochloric acid.

Preference is given to using silicon oxide-containing catalysts such as zeolites, aluminas, silicon dioxide, for example in the form of silica gel, kieselguhr or quartz, aluminum oxide, for example in the form of alpha- or gamma-aluminum oxide, and zinc oxide, boron trioxide, and also titanium dioxide. It has been found that silicon dioxide or catalysts which comprise silicon oxide components are particularly suitable.

The heterogeneous, preferably oxidic, catalysts may be arranged in a fixed bed in the reaction zone, and the vaporous mixture of esters and carrier gases can be passed over them. However, it is also possible that the catalyst is in upward and downward flowing motion (fluidized bed). Advantageously, a catalyst hourly velocity of from 0.01 to 40 g, preferably from 0.05 to 20 g, especially from 0.07 to 10 g, of reactant (mixture of 6-hydroxycaproic ester and from 0.5 to 40% by weight of adipic diester) per g of catalyst and hour is used.

The conversion to caprolactone is performed at a temperature of from 150 to 450° C., preferably at from 200 to 400° C., especially from 230 to 300° C. In general, the reaction is performed under atmospheric pressure. However, it is also possible to employ slightly reduced pressure, for example down to 500 mbar, or slightly elevated pressure, for example up to 5 bar. When a fixed bed catalyst is used, it has been found to be particularly favorable for a higher pressure to be established upstream of the catalyst than downstream of the catalyst, such that any high-boiling components which form can be deposited on the catalyst to a lesser extent, if at all.

The reaction effluent is condensed with suitable cooling apparatus. When a fixed bed catalyst is used, the reactor, for example a shaft reactor or a tube bundle reactor, can be operated in upward or downward flow mode. The reaction is effected in at least one reactor.

The reaction effluent of the cyclization comprises, as a main component, the main caprolactone product, and also the lower alcohol released in the cyclization and adipic diester, with or without unconverted 6-hydroxycaproic ester, with or without oligoester and with or without solvent. This mixture is separated by a single-stage or multistage distillation in stage 14 at reduced pressure such that caprolactone is obtained in a purity of at least 99%. The purity is preferably above 99.5%, more preferably above 99.8%.

The single-stage or multistage distillations for purifying the caprolactone are performed at bottom temperatures of from 70 to 250° C., preferably from 90 to 230° C., more preferably from 100 to 210° C., and pressures of from 1 to 500 mbar, preferably from 5 to 200 mbar, more preferably from 10 to 150 mbar.

When a column is used for this purpose, any esterification alcohol still present and other C₁ to C₆ low boilers are removed via the top, pure caprolactone is removed via a sidestream, and adipic diester and any unconverted hydroxycaproic ester which is recycled are removed via the bottom. The adipic diester may, if appropriate together with dimeric or oligomeric esters, be fed into a hydrogenation reactor and converted to 1,6-hexanediol according to WO 97/31 883 or DE-A-19750532.

When unconverted 6-hdroxycaproic ester is obtained, it is preferably passed into the distillative ester separation upstream of the caprolactone synthesis stage for recovery. It is of course also possible in principle to conduct it together with the adipic diesters into the hydrogenation to 1,6-hexanediol.

If oligomeric C₆ esters are formed, they can, according to EP-B 1 030 827, likewise be introduced into the hydrogenation to 1,6-hexanediol.

The process is illustrated in detail with reference to the examples which follow, but is in no way restricted by them.

### EXAMPLES

### Example 1

10 g/h of a mixture of 25% by weight of dimethyl adipate and 75% by weight of a methyl 6-hydroxycaproate stream which comprised 93% methyl 6-hydroxycaproate, 1.6% 1,4-cyclohexanediols, 1.4% 1,5-pentanediol, 0.3% unsaturated dimethyl adipate, 0.2% dimethyl pimelate, 1.6% dimeric esters and further compounds, each of which were present in proportions below 0.1%, prepared according to WO 97/31 883, were pumped into an evaporator at 250° C. and passed from there in gaseous form, together with 10 I (STP) of nitrogen/h at 260° C. and standard pressure over 50 ml of silicon dioxide catalyst (precipitated silica, precipitated from waterglass with sulfuric acid, 3 mm extrudates). The reaction effluent was condensed by means of a water condenser and analyzed. The methyl 6-hydroxycaproate conversion was 98%, the caprolactone selectivity based on methyl 6-hydroxycaproate was 93%, and the yield was 91%. The dimethyl adipate conversion was only approx. 10%, which led predominantly to cyclopentanone.

The collected reaction effluents were distilled batchwise in a 1 m column with random packing. At 10 mbar, it was possible to obtain caprolactone in a purity of up to 99.8%.

### Example 2

Example 1 was repeated, with the difference that the catalyst used was silicon dioxide (STR 5 mm, Davicat SMR#CCS-04-051, #03GMD363 from Grace & Comp.) and the content of dimethyl adipate was 10% by weight. A methyl 6-hydroxycaproate conversion of 56% was achieved, the caprolactone selectivity was 98% and the yield was 55%. The dimethyl adipate conversion was below 1%.

### Comparative Example 1

Example 2 from WO 97/31 883 was repeated with a hydroxycaproic acid-containing stream which, based on the total amount, comprised not 0.1% but rather approx. 5% dimethyl adipate in the feed to the liquid phase cyclization. In contrast to example 2 of WO 97/31883 without significant dimethyl adipate addition, the amount of caprolactone-containing distillate was not 1225 g, corresponding to a caprolactone yield of >90% but rather only 900 g, corresponding to a caprolactone yield of approx. 75%. The amount of bottom product was correspondingly greater.

### Comparative Example 2

Comparative example 1 was repeated, with the difference that 10% dimethyl adipate was present in the feed. The caprolactone yield was nearly 10%, the remainder consisted of oligomeric bottom product.

1. A process for preparing ε-caprolactone in a purity above 99%, which comprises cyclizing a 6-hydroxycaproic ester comprising from 0.5 to 40% by weight of adipic diester in the gas phase at from 150 to 450° C. in the presence of oxidic catalysts and obtaining ε-caprolactone from the cyclization product by distillation.

2. The process for preparing ε-caprolactone in a purity above 99% according to claim 1, wherein 6-hydroxycaproic ester comprising from 0.5 to 40% by weight of adipic diester is obtained by catalytically hydrogenating adipic diesters or reactant streams which comprise these esters as significant constitutents, distilling the hydrogenation effluent and removing the hexanediol.

3. The process for preparing ε-caprolactone in a purity above 99% according to claim 1, in which a carboxylic acid mixture which comprises adipic acid, 6-hydroxycaproic acid and small amounts of 1,4-cyclohexanediols and is obtainable as a by-product of the oxidation of cyclohexane to cyclohexanone/cyclohexanol with oxygen or oxygen-comprising gases by water extraction of the reaction mixture is esterified with a low molecular weight alcohol to the corresponding carboxylic esters, and the esterification mixture thus obtained is separated in at least one distillation stage so as to obtain the 6-hydroxycaproic ester stream comprising from 0.5 to 40% by weight of adipic diester.

4. The process for preparing ε-caprolactone in a purity above 99% according to claim 3, in which the methyl 6-hydroxycaproate comprising from 0.5 to 40% by weight of dimethyl adipate is prepared by
freeing the esterification mixture obtained of excess methanol and low boilers in a first distillation stage,
from the bottom product, in a second distillation stage, performing a separation into an ester fraction essentially free of 1,4-cyclohexanediols and a fraction comprising at least the majority of the 1,4-cyclohexanediols, and
removing the methyl 6-hydroxycaproate stream comprising from 0.5 to 40% by weight of dimethyl adipate from the ester fraction in a third distillation stage.

5. The process for preparing ε-caprolactone in a purity above 99% according to claim 1, wherein cyclization is effected in the presence of an inert carrier gas selected from nitrogen, carbon dioxide, hydrogen and noble gases.

6. The process for preparing ε-caprolactone in a purity above 99% according to claim 1, wherein silicon oxide-containing catalysts selected from zeolites, aluminas, silica gel, kieselguhr and quartz are used.

7. The process for preparing ε-caprolactone in a purity above 99% according to claim 1, wherein cyclization is effected at from 200 to 400° C.

8. The process for preparing ε-caprolactone in a purity above 99% according to claim 1, wherein cyclization is effected at from 230 to 300° C.
